(19) United States
(12) Patent Application Publication
(10) Pub. No.: US 2006/0052430 A1
(43) Pub. Date: Mar. 9, 2006

(54) Antihistamines for increased tolerance to extreme heat

(76) Inventor: Joan Smith-Sonneborn, Laramie, WY (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/217,165

(22) Filed: Sep. 1, 2005

Related U.S. Application Data
(60) Provisional application No. 60/606,840, filed on Sep. 2, 2004.

(51) Int. Cl. A61K 31/4172

(52) U.S. Cl. 514/400

(57) ABSTRACT

Histamine receptor antagonists are administered to delay the onset of or prevent heat stroke in mammals. H2 receptor antagonists including cimetidine, famotidine, nizatidine, and ranitidine are administered to the mammals prior to the exposure of the mammal to stress conditions, such as temperature, environmental, drug, exercise, or combinations thereof. The explosive temperature rise and precipitous mean arterial pressure crash seen in untreated subjects were delayed 2.6 fold (p<0.05) in H2 receptor antagonist-treated animals. The results teach that these antihistamines and acid reducers can increase tolerance to heat stress and may potentially increase survival to heat stress induced by environmental, drug related, or exercise and heat induced stress in athletes, military, and workers required to work in extreme temperature environments.

### BACKGROUND OF THE INVENTION

This invention relates generally to the prevention or delay in the onset of heat stroke and, more specifically, to the administration of antihistamines, and specifically H2 receptor antagonists, to prevent or delay the onset of heat stroke.

In the United States between 1979 and 2002, 8966 deaths were caused by exposure to excessive heat. In the summer of 2003 during and intense 19 day heat wave in France, an estimated 14,800 extra deaths were recorded due to excessive heat.

Avoiding heat disease can be as simple as cooling your environment, moving to another cooler location, or by gradual exposure to increasing temperatures. Infants or elderly may not have the option to move or may have comprised ability to acclimatize. Individuals involved in intense exertion in high heat like athletes, construction workers, or military personnel are prime candidates for heat disease. Even with the best, most advanced treatments currently available, death is often the outcome. Consequently, interventional strategies should be investigated which will allow for either the treatment of heatstroke victims or a method of delaying the onset of heatstroke, possibly allowing evacuation of a person affected by early forms of heat stress to a medical facility. Initial signs of distress can include any of the following: swelling of the hands or feet, heat rash (which may limit sweat production and lead to further illness), hyperventilation (in rare cases followed by tetany), heat cramps, or forms of syncope. These early symptoms stem from activation of standard mammalian thermoregulatory responses vasodilation for cooling, blockade or overactivation of sweat glands, panting, and loss of salt as sweat. These minor complications may lead to a faster diagnosis of heat stress and so can limit disease progression, but often these go unreported and untreated.

Heat exhaustion is a more commonly reported illness that is the result of a lowered cardiac output as the system attempts to compensate for dilated capillary beds possibly coupled with increased metabolic demand. Often heat exhaustion may be quickly reversed by administration of water and electrolytes and active cooling, but if response to cooling is slow, or if there is mental distress, then it is evidence of a more severe disease state. Exertional heat injury in vigorously active populations exposed to high temperatures that leads to differentiation between exertional heatstroke (EHS) and non-exertional heatstroke (NEHS), and is often characterized by coincident occurrence of elevated temperature and exertional rhabdomyolysis (muscle breakdown). This differentiation is important because in EHS there is a greater likelihood of tissue or organ damage prior to the onset of neurological dysfunction that is more diagnostic of heatstroke, and so may not be reported until late stages (“Heat Illness and Injury” in Heat Stress Control and Heat Casualty Management (TB. MED 507/AFPAM 48, 27-38 2003)).

Heatstroke itself is characterized by a sudden, drastic rise in core temperature (beyond 41.3° C.), occasionally accompanied by anhydrosis (loss of ability to sweat), and coma or convulsions. Other signs of hypovolemic shock are present in this stage of disease; certain changes to hemodynamic parameters are predictable.

In hypovolemic shock there are three major phases of hemodynamic modification, compensatory and decompensatory responses. If shock is reduced in some manner, recovery to normal function is termed the recompensatory phase. The compensatory phase is a period of resistance to falling blood pressure where the body attempts to maintain regular function through modification of cardiac output and vascular tone/dilation or behavior. During the compensatory phase, there is a redistribution of blood flow to allow greater dissipation of heat in the periphery. In the continuum, continued heat exposure will reverse the blood flow to thermoneutral levels reflecting the dominance of cardiovascular requirements over thermoregulation (Horowitz, M., and Samueloff, S.: Interactions between circulation and Plasma Fluids during heat stress. In Adapative Physiology to stressful environments S. Samueloff and M. Yausef eds 139-148, 1987).

If compensation fails over time, there is a precipitous drop in blood pressure—a decompensatory phase, or heat stroke. The possible movement of nucleic acids and uric acid to the blood from rhabdomyolysis, or the severe respiratory alkalosis produced by hyperventilation, and chances for organ failure rise drastically.

During heat stress, there is a diminution of blood flow to certain tissues, which may lead to tissue hypoxia, hyperventilation, and loss of acid base balance (the ratio of cation and anion concentrations in body fluids). Heat exposure can lead to acid plasma levels and metabolic acidosis due likely to lactic acid accumulation in tissues deprived of sufficient oxygen which can trigger anaerobic metabolism and lactic acid production (Horowitz and Samueloff, 1987). In a retrospective study of patients in the Saudi Arabia Heat Stroke Center, who experienced heatstroke (rectal temperature 40-43° C.), metabolic acidosis was considered to be the predominant response to heat stroke (Bouchama A, De Vol EB: Acid-base alterations in heatstroke. Intensive Care Med. 27(4):680-685. 2001). Respiratory alkalosis was also observed in the patients induced usually by hyperventilation. Hypoventilation increases the partial pressure of oxygen and can initiate respiratory acidosis while hyperventilation decreases oxygen partial pressure and initiates respiratory alkalosis. Plasma bicarbonate changes from renal mechanisms can intervene in acid base alterations (Madias N E & Adrogue H J Cross-talk between two organs: how the kidney responds to disruption of acid-base balance by the lung. Nephron Physiolol 93: 61-66. 2003) since both response are found in heat stroke patients. Therefore acid base balance is a predominant response to excessive heat.

### SUMMARY OF THE INVENTION

The invention consists of a method for ameliorating the effects of environmental, drug, exercise, or temperature-induced stress in mammals. A therapeutically effective amount of one or more histamine receptor antagonists is administered to the mammal, preferably prior to exposure of the mammal to the stress condition. In a preferred embodiment, the antagonist is an H2 receptor antagonist selected from one or more of cimetidine, famotidine, nizatidine, and ranitidine. The H2 receptor antagonist is administered in an amount between about 0.1 and about 100 mg per kilogram of body weight and preferably between about 0.25 and about 60 mg per kilogram of body weight.

The H2 receptor antagonist may be administered as a pill, tablet, or capsule, including those sold over-the-counter under the trademarks Tagamet®, Axid®, Pepcid®, and Zantac®. Alternatively, the H2 receptor antagonist may be provided in a convenient form commonly used by athletes and others exercising or working in heat-stressed environments, such as in sports bars, sports drinks, energy drinks, energy gels, powders (such as drink powders), transdermal patches, and the like.

An object of the present invention is to provide a method for preventing or delaying the onset of heat stroke in a mammal under stress conditions, such as environmental, drug, exercise or temperature-induced stress.

Another object of the present invention is to provide a method for ameliorating the effects of heat stress in a person by the administration of an antihistamine of the H2 receptor antagonist type prior to exposure to the heat stress.

Another object of the present invention is to increased tolerance in animals that must be transported and subjected to transient extreme temperature to increase their chances of survival.

A further object of the present invention is to provide a method for the prevention or delay of heat stroke in persons by the administration of antihistamines available over-the-counter in amounts within the limits of the labeling of the antihistamine.

These and other objects of the invention will be made apparent to those skilled in the art upon a review of this specification, the associated figures and the appended claims.

### BRIEF DESCRIPTION OF THE DRAWINGS

FIG. 1 is a graphical representation of internal body temperature vs. time wherein 100 percent time represents the interval from the blood pressure decline of 10 mmHg from peak value to adjust for differences in heat stroke between rats; temperature is recorded continuously in a surgically implanted computer chip. In general, the mean core body temperature rises continuously in the controls, while cimetidine pretreated animals can maintain a lower, non-pathological core temperature at least 2-4 times longer while in extreme heat.

FIG. 2 is a graphical representation of mean arterial pressure (MAP) over time wherein 100% time is the interval from exposure to heat to the drop in MAP 10 mmHg when each animal exhibited the 10 mmHg onset of heat stroke. The cimetidine treated animals MAP blood pressure crash was delayed compared to controls. The time of MAP crash coincided with the explosive rise in temperature displayed in FIG. 1.

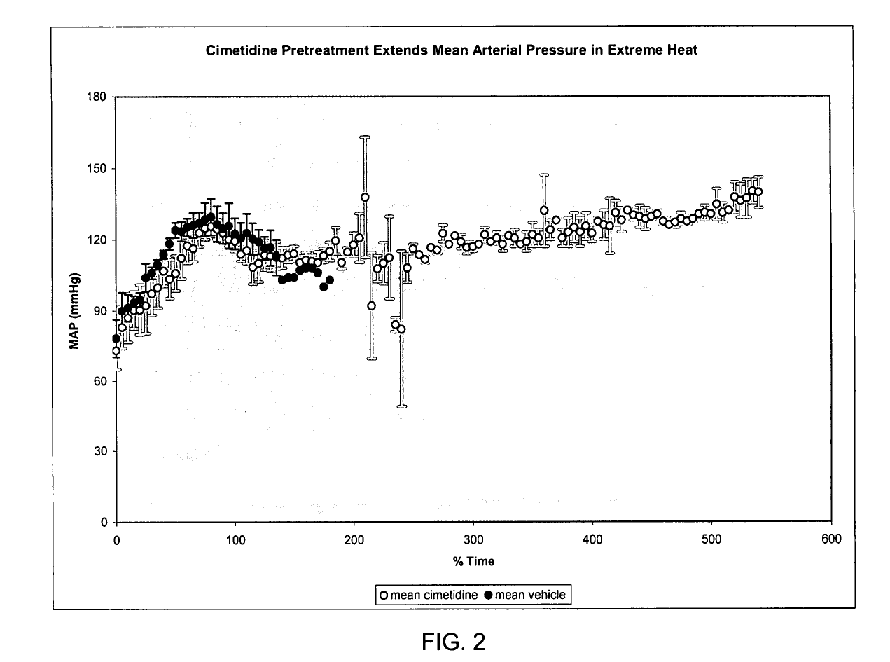

### DETAILED DESCRIPTION OF PREFERRED EMBODIMENTS

Histamine is a neurotransmitter in the central nervous system (CNS) with large numbers of histaminergic neurons in the brain and throughout the CNS (Timmerman H. Van der Groot H: New perspectives in histamine research. Agents Actions suppl 33:1-434, 1991). Histamine receptors play a roll in regulating both acid concentration of the gut, have been implicated in anxiety, activation of sympathetic nervous system, and stress-related release of hormones (Brown R E, Stevens D R, Haas H L: The physiology of brain histamine. Prog Neurobiol. April; 63(6):637-72. 2001). Additionally, histamine is well known for its role in swelling, and in the process of loss of water from vasculature to tissue. Histamine-mediated effects are mediated through four pharmacologically distinct subtypes of receptors, i.e., the H1, H2, H3 and H4 receptors, which are all members of the G-protein coupled receptor (GPCR) family.

Although H1, H2, H3 are thought to be associated with inflammatory response, gastric secretion, and neurotransmitter release respectively, identification of a new H4 histamine receptors suggests re-evaluation of the histamine receptors and their specific functions may be warranted (Jablonowski, J A, Carruthers N I, Thurmond R L. The histamine H4 receptor and potential therapeutic uses for H4 ligands. Mini Rev Med Chem 4:993-1000. 2004). The role of these histamine receptors in central mechanisms of thermoregulation is still not well understood. It is however clear that blockade of the histamine H2 receptor antagonists cimetidine or ranitidine is beneficial in heat-induced brain injury by attenuating blood-brain barrier permeability, cerebral blood flow disturbances, edema and associated cell reactions to heat (Patnaik R, Mohanty S, Sharma, H S. Blockade of histamine H2 receptors attenuate blood-brain barrier permeability, cerebral blood flow disturbances, edema formation and cell reactions following hyperthermic brain injury in the rat. Acta Neurochir Suppl. 76:535-9. 2000; Sharma H S and Cervos-Navarro J. Role of histamine in pathophysiology of heat stress in rats. Agents Actions Suppl. 33:97-102.1991; Sharma H S, Nyberg F, Cervos-Navarro J, Dey P K. Histamine modulates heat stress-induced changes in blood-brain barrier permeability, cerebral blood flow, brain oedema and serotonin levels: an experimental study in conscious young rats. Neuroscience. September;50(2):445-54, 1992).

H2 receptor antagonists as used in this specification include the antihistamines cimetidine, famotidine, nizatidine, and ranitidine. These H2 receptor antagonists are available over-the-counter under the brand names Tagamet® (SmithKline), Pepcid® (Merck), Axid® (Reliant), and Zantac® (Warner-Lambert), respectively.

Heat stress may lead to hyperventilation if strenuous work is also involved. Hyperventilation induces hypocapnia (reduced PaCO₂). Hypocapnia then initiates respiratory alkalosis, an acid-base disorder. This respiratory origination for pH distress elicits a response from the kidney in the form of a change in the amount of plasma bicarbonate (Madias and Adrogue, 2003), in a form of metabolic acidosis.

### EXAMPLE

Materials and Methods

This study was approved by the Wyoming Animal Care and Use committee in accordance with federal regulations. The experiments were performed in adherence to National Institutes of Health Guideline on the Use of Laboratory Animals. Male Sprague-Dawley® rats bred at Charles River Labs, Mass., were housed at the University of Wyoming in individual cages. The hanging steel mesh cages were held in a temperature controlled colony room with a 12:12 light/dark cycle. All rats were given ad libidum access to Purina rat chow and tap water. Rats were randomly assigned to either cimetidine (10 mg/ml) or a vehicle solute control (3% ethanol).

Rats were anestheitized with ketamine hydrochloride (0.07 mg/100 g) and 0.15 ml of xylazine. An incision was made in one hind leg and the femoral artery and vein were exposed. Catheters were constructed from a 5-cm length of PE-10 tubing that was glued into a 30 cm length of PE-50 tubing. The catheters were filled with heparinzed saline and inserted into the femoral artery and vein, tied in place with suture, superglued over the insert sites to prevent kinking and brought beneath the skin to an incision on the back of the neck, where they exited the body. Incisions were wound clipped and catheters were flushed with heperanized saline and heat-sealed until testing (McBride, S. M., Smith Sonneborn, P. Oeltgen, & F. W, Flynn: delta2 opioid receptor agonist facilitates mean arterial pressure recovery after hemorrhage in conscious rats. Shock 23:264-8, 2005). One iButton® Thermochron® (Dallas Semiconductor) was inserted next to the abdominal wall into the subdermal cavity and was programmed to record temperature at 0.5° C. precision in increments of one minute beginning on the morning following surgery.

The heating cabinet used for all conscious heat-stress experiments was constructed from a Precision Scientific Company Model 25 incubator. A standard cage was fitted with a Styrofoam sheath to fit securely against the walls of the bath, minimizing heat loss to environment. A Sunbeam 761 heating pad was placed in the incubator before insertion of the sheathed cage to generate an more even heating profile for the entire cabinet (once closed, internal temperature variation was less than 1° C. between opposing corners measured simultaneously by Thermochron®). The cabinet was closed with a Plexiglas lid with 12 holes for ventilation, a port for insertion of a thermometer, and two slots for access to catheters. The lid was secured with tape during heating. While empty and active (heating element active, heating blanket set to medium-low), the cabinet maintained a steady temperature of 44±1° C., with less than a 0.3% drop in % O₂. While active with a subject present in the cabinet, the % O₂ could fall as much as 1.5%.

Testing and Measurement

Rats were tested the day after catheterization surgery. Nine male rats were divided evenly into two groups each with catheters and iButton® Thermochrons®. The heating cabinet was activated and set to 44±1° C. Thirty minutes before heat, animals were connected to the arterial and venous catheters and were monitored for five minutes for positive pressure in the arterial catheter (AcqKnowledge® version 3.7), after which they were given an injection of 0.2 ml per 10 g of either the H2 antagonist cimetidine dissolved in 3% by volume ethanol (EtOH) or the 3% by volume EtOH solvent as a control. After thirty minutes, animals were placed in the heating cabinet and recording of hemodynamic parameters began using the AcqKnowledge® system. MAP (Mean Arterial Pressure) was monitored through peak value and to 40 mmHg, when the animal was removed from the cabinet and given access to food and water. Survival was recorded.

Data were expressed as a mean value±SEM (Standard Error of the Mean) and data was analyzed using SPSS® software with a P value of <0.05 considered significant. To compensate for differences in heat sensitivity of animals, homodynamic comparisons between animals were made by setting 100% time as the moment at which the mean arterial pressure began to decrease from the peak value taken as the onset of heat stroke (Kao, K., Chio, T. Y. C. C., Lin M. T.: Hypothalmic dopamine release and local cerebral flow during onset of heatstroke in rats. Stroke 25 2483-2487, 1994; Chiu W T, Kao T Y, Lin M T, Increased survival in experimental rat heatstroke by continuous perfusion of interleukin-1 receptor antagonist Neuroscience Res 24: 159-163, 1996).

Results

Pretreatment with cimetidine more than doubled (2.6 fold) the interval between initiation of exposure to extreme heat and heat stroke crash to 40 mmHg (P<0.05) (Table 1).

TABLE 1
Cimetidine Pretreatment Effects on Induced Tolerance to Heat Stroke in Conscious Rats*

| Group | | Heat Tolerance** | Max. T_{B} | Max. HR | Max. MAP |
|---|---|---|---|---|---|
| Animals | Number | minutes ± SEM | in ° C. ± SEM. | in BPM ± SEM. | in mmHg ± SEM. |
| Control Vehicle | 4 | 126 ± 25 | 40.9 ± 0.7 | 546 ± 10 | 134 ± 7 |
| Cimetidine | 5 | 315 ± 61*** | 41.9 ± 0.9 | 532 ± 28 | 173 ± 23 |

*Rats were intravenously injected with cimetidine (10 mg/kg in 3% EtOH) or an equal volume of vehicle (3% EtOH) 30 minutes prior to heat exposure at 44° C. ± 1 and monitored until onset of heat shock. T_{B} (body temperature), HR (heart rate), BPM (beats per minute) and MAP (mean arterial pressure).
**Heat Tolerance is defined as the time of heat exposure until heat shock, when core body temperature is T_{B} ≧ 41.3° C. and MAP is <40 mmHg.
***Significantly greater heat tolerance interval than control group, determined by Student's T-test with p-value less than 0.05.

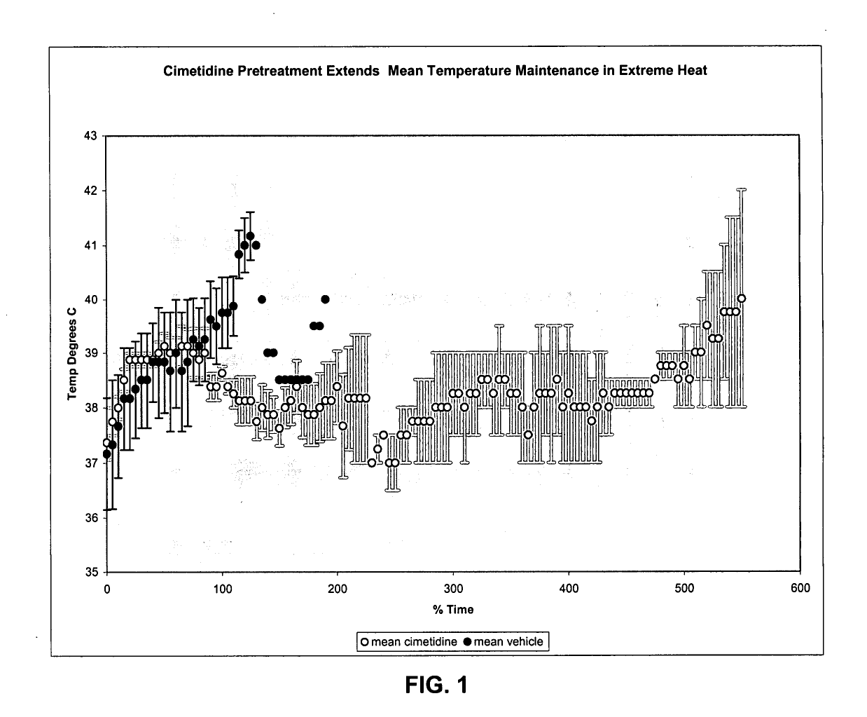

Cimetidine pretreated animals maintained a lower core body temperature after onset of heat stroke (defined as a MAP drop of 10 mmHg from peak values) until the explosive rise in temperature and heat shock (defined as a MAP crash to 40 mmHg) than controls (FIG. 1). Both vehicle controls and cimetidine-treated animals were able to maintain MAP until a precipitous drop in MAP and were not significantly different except in the delayed time until blood pressure crash. Heart rate showed large variations within the experimental groups and did not differ in the interval from exposure to heat stroke, MAP crash.

Discussion

The data presented shows that pretreatment with an H2 receptor antagonist, specifically the antihistamine and acid reducer cimetidine, indeed showed a significant increase in tolerance to extreme heat in conscious rats. The time from onset on exposure to onset of heat stroke and onset of heat shock blood pressure crash was increased 2.6 fold allowing extended time in extreme heat before irreversible pathophysiological symptoms ensued showing increased tolerance in cimetidine pretreated animals in extreme heat despite continuous heat exposure. Cimetidine apparently blocked H2-related responses that interfered with the usual pathological progression in heat shock. This finding of this study implies that pretreatment of animals with cimetidine could increase survival to heat in situations when escape from the heat is not an option (eg., infants, elderly, power outage, transportation of animals, workers in heated environments, emergency evacuations, and rescue dogs). Cimetidine apparently blocked H2-related responses that interfered with the usual myriad of pathological progressions in heat stroke, presumably by interference in histamine related cascades and/or in pathologies associated with acidosis. This study confirms previous studies (Patnaik et al., 2000) that the H2 histamine receptor is a major factor in pathological hyperthermic response but shows for the first time that an H2 receptor antagonist, cimetidine can increase tolerance to heat in animals continuously exposed to the heated environment. The waning effect of cimetidine may reflect the limited half-life of cimetidine of 2 hours, which may be compensated by additional treatments.

Although these results were obtained using intravenous injection, stress-induced modulations were reduced in chickens by feeding cimetidine in the diet, though the stress was not heat (Grbarevic Z, Dzaja P, Peric J Seman V Bidin Z, Mazija H. Mas N, Mikulec Z Culjak, Simec Z, Najari B. Effects of cimetidine on broiler fattening and on stress induced gizzard erosion in chicken Acta Vet Hung. 47:233-241 1999). The use of cimetidine in Sports drinks, supplements, power bars or tablets will likely be as effective as injections since these H2 anatagonists are approved as anti-acid medications. Metabolic acidosis is a predominant pathophysiological response to heat that can be ameliorated by H2 antagonists.

Previous studies indicated that ranitidine was even more effective than cimetidine in attenuating the blood brain barrier permeability in extreme heat. Therefore ranitidine is also a likely candidate drug for heat tolerance.

The ability of nonsteroidal anti-inflammatory agents widely used for inflammatory reactions and disease has have been shown to induce heat shock factor-1 (HSF-1) at lower than usual threshold temperatures providing heat shock protein (HSP) protection against cytotoxic damage. Alteration of histamine release and increased HSP could work synergistically in tolerance to extreme heat.

Histamine modulated release of inflammatory cytokines (Marone G, Granata F, Spadaro G, Genovese A, Triggiani M. 2003 The histamine-cytokine network in allergic inflammation J Allergy Clin Immunol. October 2003; 112(4 Suppl):S83-8) may have a role in the tolerance to heat found here, but previous studies indicate that H1, not H2 receptors are involved in the cytokine response.

Theoretically, cimetidine effects seen previously in reduction of brain edema and breakdown of the blood brain barrier (Sharma et al., 1991; Sharma et al., 1992; Patnaik et al., 2000) could be explained by inhibition of histamine-induced phosphorylation of intracellular adhesion protein VE cadeherin found in the vascular epithelium and “activation of the gaps between cells which could allow blood and fluid to seep into the extracellular space (Andriopoulou P, Navarro P, Zanetti A, Lampugnani M G, Dejana E. Histamine induces tyrosine phosphorylation of endothelial cell-to-cell adherens junctions. Arterioscler Thromb Vasc Biol. October 1999; 19(10):2286-97, 1999). Alternatively, it has been suggested that histamine may have a role in nitric oxide release and permeability of the blood brain barrier (Mayham W G: Role of nitric oxide in histamine induced increase in permeability of blood brain barrier. Brain Research 743:70-76, 1996) and this effect decreased by the H2 antagonist.

Interestingly, heated rats pretreated simultaneously with H1 and H2 antagonists using microwave heat did not show any improvement in survival (Jauchem J. R., Ryan, K. L. & Tehrany M. R. Effects of histamine receptor blockade on cardiovascular changes induced by 35 GHz radio frequency radiation heating. Autonomic & Autacoid Pharmacology 24::1-17. 2004). Since our results show the significant increase in tolerance to heat with blockade of only the H2 receptor, the H2 receptor effect may be neutralized in combination with the H1 antagonist or microwave heat may present a different cascade of pathological symptoms than environmental heat. Our results imply that H2 antagonists represent a potential pharmaceutical intervention in heat stroke in animals and may offer increased tolerance to humans or animals exposed to extreme heat and delay heat shock.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

What is claimed is:

1. A method for ameliorating the effects of environmental, drug, exercise, or temperature-induced stress in mammals, comprising the step of administering a therapeutically effective amount of one or more histamine receptor antagonists.

2. A method for ameliorating the effects of heat-induced stress in mammals, comprising the step of administering a therapeutically effective amount of one or more H2 receptor antagonists.

3. The method of claim 2, wherein the H2 receptor antagonist is selected from the group consisting of cimetidine, famotidine, nizatidine, and ranitidine.

4. A method as defined in claim 2 wherein the amount of the H2 receptor antagonist is between about 0.1 and about 100 mg per kilogram of body weight.

5. A method as defined in claim 2 wherein the amount of the H2 receptor antagonist is between about 0.25 and about 60 mg per kilogram of body weight.

6. A method as defined in claim 1, wherein the histamine receptor antagonist is administered prior to exposure of the mammal to stress conditions.

7. A method for ameliorating the effects of environmental, drug, exercise, or temperature-induced stress in humans, comprising the step of administering one or more over-the-counter histamine receptor antagonists in an amount within the labeling limits of the histamine receptor antagonist.

8. A method as defined in claim 7, wherein the antagonist is selected from the group consisting of Axid®, Pepcid®, Tagamet®, and Zantac®.

9. A method as defined in claim 1, wherein the H2 receptor antagonist is administered in the form selected from the group comprising pills, tablets, capsules, bars, drinks, gels, powders, and transdermal patches.
